(19) United States
(12) Plant Patent Application Publication
(10) Pub. No.: US 2005/0177912 P1
(43) Pub. Date: Aug. 11, 2005

(54) Chrysanthemum plant named 'Sunbutton Yellow'

(50) CHRYSANTHEMUM MORIFOLIUM; CHRYSANTHEMUM

(76) Inventor: Keith G. Lintott, Bognor Regis (GB)

Correspondence Address: Mark K. Dickson, Suites 200 & 300, 2941 Fairview Park Drive, Falls Church, VA, 22042, US

(21) Appl. No.: 11/055,230

(22) Filed: Feb. 9, 2005

Related U.S. Application Data
(60) Provisional application No. 60/543,817, filed on Feb. 11, 2004.

(51) Int. Cl. B60B 17/00; A01H 5/00

(52) U.S. Cl. PLT/295

(57) ABSTRACT

A new variety of Chrysanthemum plant named ‘Sunbutton Yellow’ having a domed capitulum of small yellow anemone flowers with a darker cushion and a round shape.

This application claims the benefit of U.S. Provisional Application No. 60/543,817, filed on Feb. 11, 2004.

### LATIN NAME OF THE GENUS AND SPECIES

Botanical classification: Chrysanthemum morifolium.

### VARIETY DENOMINATION

The new Chrysanthemum variety denomination is ‘Sunbutton Yellow.’

### BACKGROUND OF THE INVENTION

The present invention comprises a new and distinct cultivar of Chrysanthemum botanically known as Chrysanthemum morifolium, and referred to by the variety name ‘Sunbutton Yellow.’

‘Sunbutton Yellow,’ identified as 30924-0, originated from a cross, made in a controlled breeding program in Chichester, West Sussex, United Kingdom. The female parent of the new Chrysanthemum variety is an undistributed, unpatented seedling identified as ‘G15E2.’ The male parent of the new Chrysanthemum variety is the variety known as ‘Leyla.’ The new variety ‘Sunbutton Yellow’ has been asexually reproduced, by vegetative cuttings in Chichester, West Sussex, United Kingdom and the distinguishing characteristics are retained through successive generations of asexual reproduction.

### BRIEF SUMMARY OF THE INVENTION

‘Sunbutton Yellow’ is a garden type of Chrysanthemum plant variety having a domed round canopy of small, yellow anemone type flowers that have a dark reddish center.

### Comparison with Female Parent

Plants of the new Chrysanthemum variety ‘Sunbutton Yellow’ are dissimilar to the parent variety ‘G15E2’ in plant habit and growth rate. In side-by-side comparisons in Chichester, West Sussex, United Kingdom, under commercial practice, plants of the new Chrysanthemum variety ‘Sunbutton Yellow’ differed from plants of the parent variety ‘G15E2’ in the following characteristics.

1. The new Chrysanthemum variety ‘Sunbutton Yellow’ produces small yellow anemone type flowers with a dark reddish center whereas the parent variety ‘G15E2’ produces pale pink small anemone type flowers.

2. Plants of the new Chrysanthemum variety ‘Sunbutton Yellow’ have smaller and shorter inflorescence than plants of the parent variety ‘G15E2.’

3. Plants of the new Chrysanthemum variety ‘Sunbutton Yellow’ are less vigorous than plant of the parent variety ‘G15E2.’

### Comparison with Male Parent

Plants of the new Chrysanthemum variety ‘Sunbutton Yellow’ are dissimilar to the variety ‘Leyla’ in plant habit and growth rate. In side-by-side comparisons in Chichester, West Sussex, United Kingdom, under commercial practice, plants of the new Chrysanthemum variety ‘Sunbutton Yellow’ differed from plants of the parent variety ‘Leyla’ in the following characteristics.

1. The new Chrysanthemum variety ‘Sunbutton Yellow’ produces small yellow anemone type flowers with a dark reddish center whereas the parent variety ‘Leyla’ produces small yellow, single type flowers.

2. Plants of the new Chrysanthemum variety ‘Sunbutton Yellow’ have smaller and shorter inflorescence than plants of the parent variety ‘Leyla.’

3. Plants of the new Chrysanthemum variety ‘Sunbutton Yellow’ are less vigorous than plants of the parent cultivar ‘Leyla.’

### Comparison with Other Variety

Plants of the new Chrysanthemum variety ‘Sunbutton Yellow’ are dissimilar to the unpatented variety ‘Boulevard Yellow’ in plant habit and growth rate. In side-by-side comparisons in Chichester, West Sussex, United Kingdom, under commercial practice, plants of the new Chrysanthemum variety ‘Sunbutton Yellow’ differed from plants of the variety ‘Boulevard Yellow’ in the following characteristics.

1. The new Chrysanthemum variety ‘Sunbutton Yellow’ produces small yellow anemone type flowers with a dark reddish center whereas the variety ‘Boulevard Yellow’ produces slightly larger yellow anemone flowers with a yellow center.

2. Plants of the new Chrysanthemum variety ‘Sunbutton Yellow’ are less vigorous than plants of the variety ‘Boulevard Yellow.’

3. Plants of the new Chrysanthemum variety ‘Sunbutton Yellow’ flower faster than plants of the variety ‘Boulevard Yellow’ under natural season conditions.

### BRIEF DESCRIPTION OF ILLUSTRATION

Typical specimens of the plant and flowers for the new Chrysanthemum variety ‘Sunbutton Yellow’ are shown in the accompanying digital photograph. The colors shown are as true as possible within the usual limits of this kind of illustration.

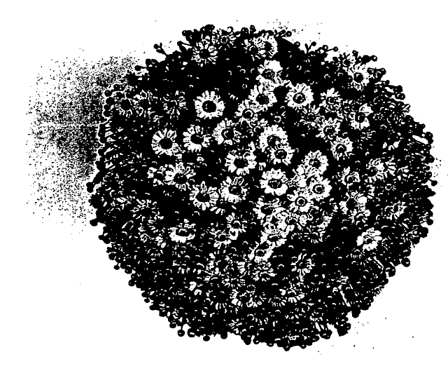

FIG. 1 is a whole plant view of the new variety ‘Sunbutton Yellow’ grown in a pot. The plant shown in the illustration is 63 days from the commencement of planting outdoors in mid-June.

### DETAILED BOTANICAL DESCRIPTION

The following description of the new Chrysanthemum variety ‘Sunbutton Yellow’ is of plants grown outside, natural season, in Chichester, West Sussex, United Kingdom in the month of June. The variety has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 50° F. at night and about 60° F. to 75° F. during the day under natural light conditions. The plants were grown outdoors in mid-June in 4 liter pots using loamless compost. No growth retardants were used. To produce a commercial product the plants may be pinched once with the center bud removed. The typical container size for commercial growth is 4 liters (8″). It has been observed that the shelf life of the new variety is about 26 days with a response time of about 9 weeks natural season. The new variety is suitable for growth in a temperature range of 50° F. to 75° F.

The new Chrysanthemum variety may be produced as a pot plant. The following description is with respect to a plant produced as a garden pot plant. In the description of this new Chrysanthemum variety, color values have been taken from The Royal Horticultural Society Colour Chart (R.H.S.C.C.).

### PLANT

Plant type: Pot.
Habit: Domed-Round.
Height: 45 cm-55 cm.
Width: 45 cm-50 cm.
Branching characteristics: Free branching and spreading.
Length of lateral branches: 9 cm-15 cm.
Number of breaks from pinch: 7-12.
Stem:
   Length.—8 cm-9 cm.
   Diameter.—0.6 cm-0.8 cm.
   Texture.—Rough.
   Color.—near 137A.
Response time: near 63 days natural season.
Vigor: Medium.
Shelf life: near 26 days.
Disease (susceptibility/resistance observed): None observed to date.
Pest (susceptibility/resistance observed): None observed to date.
Drought and temperature (susceptibility/resistance observed): None observed to date.
Growth retardant type and treatment: No treatment necessary. The plants were grown for 9 weeks in natural season conditions.
Propagation:
   Type.—Vegetative Propagation via Stem Cuttings.
   Time to rooting.—12 days with soil temperatures of 70° F.
   Rooting habit.—Fine and Fibrous.

### FOLIAGE

Number of leaves per lateral branch: 7-15.
Compound or single: Single,
Arrangement of leaves: Alternate.
   Shape of leaf.—Typically 5 lobed ovate.
   Size of leaf.—Width: 1.1 cm-2.4 cm Length: 1.9 cm-3.5 cm.
   Leaf apex.—Acute.
   Base.—Obtuse.
   Attachment.—Petioled.
   Aspect.—Slightly undulating.
   Margin.—Palmately lobed.
   Surface characteristics.—Top: Slightly pubescent Bottom: Slightly pubescent.
Petiole:
   Color.—near 137B.
   Length.—0.6 cm-0.8 cm.
Venation: Net. Prominent mid vein at underside.
   Color.—upper side: near 144B under side: near 144B.
Color:
   Mature leaf, upper side.—near 138A; under side: near 138B.
   Young leaf, upper side.—near 138A; underside: near 138B.

### FLOWER

Flower appearance: Matte.
Flower type: Anemone.
Flower form: Flat.
Flower shape: Round.
Flowering habit: Cyme.
Number of blossoms per branch: 12-20.
Inflorescence form: Corymbiform.
   Depth of fully expanded blossoms.—0.8 cm-1.2 cm.
   Diameter of fully expanded blossoms.—2.5 cm-3.5 cm.
   Anemone center diameter.—0.8 cm-1.0 cm.
Phyllaries:
   Number.—22-25.
   Color.—near 137C.
   Length.—0.4 cm-0.7 cm.
   Width.—0.1 cm-0.2 cm.
   Texture/Appearance.—Dull/Pubescent.
Peduncle: Peduncle length is shortest at the terminal flower and progressively longer down the stem; longest length is on average 12 cm, shortest length is on average 7 cm.
   Length.—7 cm-12 cm.
   Color.—near 137C.
   Surface.—Slightly Pubescent.
   Habit.—Slightly Curving.
   Strength.—Medium.
Pedicel:
   Length.—terminal: 5 cm-6 cm; lateral: 7 cm-10 cm.
   Color.—near 137C.
   Surface.—Slightly pubescent.
   Habit.—Slightly curving.
   Strength.—Medium.
Ray florets:
   Form/Shape.—Straight.
   Texture/Appearance.—Matte.
   Number per flower.—28-34.
   Length.—1.5 cm-1.9 cm; Width: 0.4 cm-0.6 cm.
   Apex.—Rounded slightly dentate.
   Base.—Tubular.
   Margin.—Entire.
Disc florets:
   Form/Shape.—Cylindrical.
   Texture/Appearance.—Shiny.
   Number per flower.—120-150.
   Length.—0.6 cm-0.8 cm: Width: 0.1 cm.
   Diameter of disc.—0.7 cm-1.0 cm.
Fragrance: Slight.
Flower bud (at onset of color):
   Length.—0.8 cm.
   Diameter.—0.8 cm.
   Form/shape.—Ball shaped.
General flower color:
   1. Ray florets, upper side.—Immature: near 17A Mature: near 14B Older/Fading: near 14C.
   2. Ray florets, under side.—Immature: near 17A Mature: near 14B Older/Fading: near 14C.
   3. Disc florets, outer.—Immature: near 13A Mature: near 13B Older/Fading: near 13B.
   4. Disc florets, inner.—Immature: near 45B Mature: near 45C Older/Fading: near 45C.
   5. Bud.—137B.
Flower progression with age; Flowers fade slightly with age.
Lastingness of blooms is about 26 days.

### REPRODUCTIVE ORGANS

Gynoecium present on Ray and Disc florets.
Ray florets per individual flower:
   Pistil member.—28-34 observed.
   Stigma color.—near 144D.
   Stigma shape.—Forked.
   Style color.—near 144D.
   Style length.—0.1 cm-0.2 cm.
Disc florets per individual flower:
   Pistil number.—80-90 observed.
   Stigma color.—near 144D.
   Stigma shape.—Forked.
   Style color.—near 144D.
   Style length.—0.2 cm-0.3 cm.
Androecium present on Disc florets only.
Disc florets per individual flower:
   Stamen number.—50-60 observed.
   Anther shape.—Ovoid.
   Anther color.—near 14C.
   Pollen color.—near 12A.
   Pollen quantity.—Small.
Fruit and seeds: None observed.

1. A new and distinct variety of Chrysanthemum plant, substantially as described and illustrated herein.
